(19) United States
(12) Patent Application Publication
(10) Pub. No.: US 2015/0106027 A1
(43) Pub. Date: Apr. 16, 2015

(54) CHARACTERIZATION OF CRUDE OIL BY NMR SPECTROSCOPY

(71) Applicants: Omer Refa Koseoglu, Dhahran (SA); Adnan Al-Hajji, Dammam (SA); Mohammed Abdullah Al-Ghamdi, Dhahran (SA); Alexander Rebrov, Dhahran (SA)

(72) Inventors: Omer Refa Koseoglu, Dhahran (SA); Adnan Al-Hajji, Dammam (SA); Mohammed Abdullah Al-Ghamdi, Dhahran (SA); Alexander Rebrov, Dhahran (SA)

(21) Appl. No.: 13/397,273

(22) Filed: Feb. 15, 2012

Related U.S. Application Data
(60) Provisional application No. 61/445,175, filed on Feb. 22, 2011.

(51) Int. Cl. G01N 24/08; G06F 19/00; G01N 33/28; G01R 33/46

(52) U.S. Cl. CPC G01N 24/081; G01N 33/28
USPC 702/23; 702/25

(57) ABSTRACT

A system and a method for applying ¹³C or ¹H NMR spectroscopy to a sample of crude oil in order to calculate the cetane number, pour point, cloud point, aniline point and octane number of a gas oil fraction of the crude oil.

### RELATED APPLICATION

This application claims the benefit of U.S. Provisional Patent Application No. 61/445,175 filed Feb. 22, 2011, the disclosure of which is hereby incorporated by reference.

### FIELD OF THE INVENTION

This invention relates to a method and process for the evaluation of samples of crude oil and its fractions by nuclear magnetic resonance (NMR) spectroscopy, avoiding the need to conduct crude oil assays.

### BACKGROUND OF THE INVENTION

Crude oil originates from the decomposition and transformation of aquatic, mainly marine, living organisms and/or land plants that became buried under successive layers of mud and silt some 15-500 million years ago. They are essentially very complex mixtures of many thousands of different hydrocarbons. Depending on the source, the oil predominantly contains various proportions of straight and branched-chain paraffins, cycloparaffins, and naphthenic, aromatic, and polynuclear aromatic hydrocarbons. These hydrocarbons can be gaseous, liquid, or solid under normal conditions of temperature and pressure, depending on the number and arrangement of carbon atoms in the molecules.

Crude oils vary widely in their physical and chemical properties from one geographical region to another and from field to field. Crude oils are usually classified into three groups according to the nature of the hydrocarbons they contain: paraffinic, naphthenic, asphaltic, and their mixtures. The differences are due to the different proportions of the various molecular types and sizes. One crude oil can contain mostly paraffins, another mostly naphthenes. Whether paraffinic or naphthenic, one can contain a large quantity of lighter hydrocarbons and be mobile or contain dissolved gases; another can consist mainly of heavier hydrocarbons and be highly viscous, with little or no dissolved gas. Crude oils can also include heteroatoms containing sulfur, nitrogen, nickel, vanadium and other elements in quantities that impact the refinery processing of the crude oil fractions. Light crude oils or condensates can contain sulfur in concentrations as low as 0.01 W %; in contrast, heavy crude oils can contain as much as 5-6 W %. Similarly, the nitrogen content of crude oils can range from 0.001-1.0 W %.

The nature of the crude oil governs, to a certain extent, the nature of the products that can be manufactured from it and their suitability for special applications. A naphthenic crude oil will be more suitable for the production of asphaltic bitumen, a paraffinic crude oil for wax. A naphthenic crude oil, and even more so an aromatic one, will yield lubricating oils with viscosities that are sensitive to temperature. However, with modern refining methods there is greater flexibility in the use of various crude oils to produce many desired type of products.

A crude oil assay is a traditional method of determining the nature of crude oils for benchmarking purposes. Crude oils are subjected to true boiling point (TBP) distillations and fractionations to provide different boiling point fractions. The crude oil distillations are carried out using the American Standard Testing Association (ASTM) Method D 2892. The common fractions and their nominal boiling points are given in Table 1.

TABLE 1

| Fraction | Boiling Point, ° C. |
|---|---|
| Methane | −161.5 |
| Ethane | −88.6 |
| Propane | −42.1 |
| Butanes | −6.0 |
| Light Naphtha | 36-90 |
| Mid Naphtha | 90-160 |
| Heavy Naphtha | 160-205 |
| Light gas Oil | 205-260 |
| Mid Gas Oil | 260-315 |
| Heavy gas Oil | 315-370 |
| Light Vacuum Gas Oil | 370-430 |
| Mid Vacuum Gas Oil | 430-480 |
| Heavy vacuum gas oil | 480-565 |
| Vacuum Residue | 565+ |

The yields, composition, physical and indicative properties of these crude oil fractions, where applicable, are then determined during the crude assay work-up calculations. The compositional and property information obtained in a crude oil assay is given in Table 2,

TABLE 2

| Property | Unit | Property Type | Fraction |
|---|---|---|---|
| Yield Weight and Volume % | W % | Yield. | All |
| API Gravity | ° | Physical | All |
| Viscosity Kinematic @ 38° C. | ° | Physical | Fraction boiling >250° C. |
| Refractive Index @ 20° C. | Unitless | Physical | Fraction boiling <400° C. |
| Sulfur | W % | Composition | All |
| Mercaptan Sulfur, W % | W % | Composition | Fraction boiling <250° C. |
| Nickel | ppmw | Composition | Fraction boiling >400° C. |
| Nitrogen | ppmw | Composition | All |
| Flash Point, COC | ° C. | Indicative | All |
| Cloud Point | ° C. | Indicative | Fraction boiling >250° C. |
| Pour Point, (Upper) | ° C. | Indicative | Fraction boiling >250° C. |
| Freezing Point | ° C. | Indicative | Fraction boiling >250° C. |
| Microcarbon Residue | W % | Indicative | Fraction boiling >300° C. |
| Smoke Point, mm | mm | Indicative | Fraction boiling between |
| | | | 150-250 |
| Octane Number | Unitless | Indicative | Fraction boiling >250° C. |
| Cetane Index | Unitless | Indicative | Fraction boiling between |
| | | | 150-400 |
| Aniline Point | ° C. | Indicative | Fraction boiling >520° C. |

Due to the number of distillation cuts and the number of analyses involved, the crude oil assay work-up is both costly and time consuming.

In a typical refinery, crude oil is first fractionated in the atmospheric distillation column to separate sour gas and light hydrocarbons, including methane, ethane, propane, butanes and hydrogen sulfide, naphtha (36°-180° C.), kerosene (180°-240° C.), gas oil (240°-370° C.) and atmospheric residue (>370° C.). The atmospheric residue from the atmospheric distillation column is either used as fuel oil or sent to a vacuum distillation unit, depending on the configuration of the refinery. The principal products obtained from vacuum distillation are vacuum gas oil, comprising hydrocarbons boiling in the range 370°-520° C., and vacuum residue, comprising hydrocarbons boiling above 520° C. The crude assay data help refiners to understand the general composition of the crude oil fractions and properties so that the fractions can be processed most efficiently and effectively in an appropriate refining unit. Indicative properties are used to determine the engine/fuel performance or usability or flow characteristic or composition. A summary of the indicative properties and their determination methods with description are given below.

The cetane number of diesel fuel oil, determined by the ASTM D613 method, provides a measure of the ignition quality of diesel fuel; as determined in a standard single cylinder test engine; which measures ignition delay compared to primary reference fuels. The higher the cetane number; the easier the high-speed; direct-injection engine will start; and the less white smoking and diesel knock after start-up are. The cetane number of a diesel fuel oil is determined by comparing its combustion characteristics in a test engine with those for blends of reference fuels of known cetane number under standard operating conditions. This is accomplished using the bracketing hand wheel procedure which varies the compression ratio (hand wheel reading) for the sample and each of the two bracketing reference fuels to obtain a specific ignition delay, thus permitting interpolation of cetane number in terms of hand wheel reading.

The octane number, determined by the ASTM D2699 or D2700 methods, is a measure of a fuel's ability to prevent detonation in a spark ignition engine. Measured in a standard single-cylinder; variable-compression-ratio engine by comparison with primary reference fuels. Under mild conditions, the engine measures research octane number (RON), while under severe conditions, the engine measures motor octane number (MON). Where the law requires posting of octane numbers on dispensing pumps, the antiknock index (AKI) is used. This is the arithmetic average of RON and MON, (R+M)/2. It approximates the road octane number, which is a measure of how an average car responds to the fuel.

The cloud point, determined by the ASTM D2500 method, is the temperature at which a cloud of wax crystals appears when a lubricant or distillate fuel is cooled under standard conditions. Cloud point indicates the tendency of the material to plug filters or small orifices under cold weather conditions. The specimen is cooled at a specified rate and examined periodically. The temperature at which cloud is first observed at the bottom of the test jar is recorded as the cloud point. This test method covers only petroleum products and biodiesel fuels that are transparent in 40 mm thick layers, and with a cloud point below 49° C.

The pour point of petroleum products, determined by the ASTM D97 method, is an indicator of the ability of oil or distillate fuel to flow at cold operating temperatures. It is the lowest temperature at which the fluid will flow when cooled under prescribed conditions. After preliminary heating, the sample is cooled at a specified rate and examined at intervals of 3° C. for flow characteristics. The lowest temperature at which movement of the specimen is observed is recorded as the pour point.

The aniline point, determined by the ASTM D611 method, is the lowest temperature at which equal volumes of aniline and hydrocarbon fuel or lubricant base stock are completely miscible. A measure of the aromatic content of a hydrocarbon blend is used to predict the solvency of a base stock or the cetane number of a distillate fuel. Specified volumes of aniline and sample, or aniline and sample plus n-heptane, are placed in a tube and mixed mechanically. The mixture is heated at a controlled rate until the two phases become miscible. The mixture is then cooled at a controlled rate and the temperature at which two phases separate is recorded as the aniline point or mixed aniline point.

To determine these properties of gas oil or naphtha fractions conventionally, these fractions have to be distilled off from the crude oil and then measured/determined using various analytical methods that are laborious, costly and time consuming.

Nuclear magnetic resonance (NMR) is a property that magnetic nuclei have under a magnetic field and applied electromagnetic (EM) pulse or pulses, which causes the nuclei to absorb energy from the EM pulse and radiate this energy back out. The energy radiated back out is at a specific resonance frequency which depends on the strength of the magnetic field and other factors. This allows the observation of specific quantum mechanical magnetic properties of an atomic nucleus. Many scientific techniques exploit NMR phenomena to study molecular physics, crystals and non-crystalline materials through NMR spectroscopy. Nuclear magnetic resonance spectroscopy, most commonly known as NMR spectroscopy, is a technique which exploits the magnetic properties of certain nuclei.

All stable isotopes that contain an odd number of protons and/or of neutrons have an intrinsic magnetic moment and angular momentum, in other words a nonzero spin, while all nuclides with even numbers of both have spin 0. The most commonly studied nuclei are ¹H (the most NMR-sensitive isotope after the radioactive ³H) and ¹³C, although nuclei from isotopes of many other elements (e.g. ²H, ¹⁰B, ¹¹B, ¹⁴N, ¹⁵N, ¹⁷O, ¹⁹F, ²³Na, ²⁹Si, ³¹P, ³⁵Cl, ¹¹³Cd, ¹²⁹Xe, ¹⁹⁵Pt) are studied by high-field NMR spectroscopy as well.

NMR is a technique for determining the structure of organic compounds. NMR is non-destructive, and with modern instruments good data can be obtained from samples weighing less than a milligram. When a sample is placed in a magnetic field, NMR active nuclei (such as ¹H or ¹³C) absorb at a frequency characteristic of the isotope. The resonant frequency, energy of the absorption and the intensity of the signal are proportional to the strength of the magnetic field. For example, in a 21 tesla magnetic field, protons resonate at 900 MHz. It is common to refer to a 21 T magnet as a 900 MHz magnet, although different nuclei resonate at a different frequency at this field strength.

The currently used crude oil assay method is costly in terms of money and time. It costs about $50,000 US and takes two months to complete one assay. With our proposed method, the crude oil can be classified easily by NMR and/or density data and many decisions can be made for purchasing and/or processing.

Any new rapid, direct method to help better understand the crude oil composition and properties from the analysis of whole crude oil will save producers, marketers, refiners and/or other crude oil users substantial expense, effort and time. Therefore, a need exists for an improved system and method for determining the properties of crude oil fractions from different sources and classifying the crude oil fractions based on their boiling point characteristics and/or properties.

### SUMMARY OF THE INVENTION

The above objects and further advantages are provided by the present invention which broadly comprehends a system and a method for determining the indicative properties of a crude oil sample. In accordance with the invention, indicative properties (i.e., cetane number, pour point and cloud point, aniline point and octane number) of gas oil fraction in crude oils are predicted by a direct NMR Spectroscopy measurement of crude oils. The correlations also provide information about the gas oil properties without fractionation/distillation (crude oil assays) and will help producers, refiners, and marketers to benchmark the oil quality and, as a result, valuate the oils without performing the customary extensive and time-consuming crude oil assays.

### BRIEF DESCRIPTION OF THE DRAWINGS

Further advantages and features of the present invention will become apparent from the following detailed description of the invention when considered with reference to the accompanying drawing which is a graphic plot of ¹³C NMR data for the oils in a crude oil sample solution prepared as described below.

### DETAILED DESCRIPTION OF INVENTION

Crude oil solutions were analyzed by ¹³C and ¹H NMR spectrometry. The quantitative NMR spectra were recorded at room temperature on a Varian VNMS 500 NMR spectrometer operating at 499.78 MHz for ¹H and 125.67 MHz for ¹³C, respectively, using Dual Broadband SW/PFG probe with 5 mm 506-PP (Wilmad Glass CO., Inc.) NMR sample tubes. The NMR experiments were carried out using 40% w/v sample solution in deuterated chloroform (99.8% D, Cambridge Isotope Laboratories Inc.) with tetramethylsilane (TMS) used as an internal standard. ¹H was performed using 16 scan numbers, 45 degree pulse length of 4.75 us, 5 s relaxation delay, 3 s acquisition time, 10 K time domain data, 15060 Hz spectra width and, 64 repetitions.

A quantitative ¹³C experiment was performed and an inverse gated WALTZ-16 modulated decoupling mode was used to suppress nuclear Overhauser enhancement. The experimental parameter were: 30 degree pulse length of 2.7 us with a relaxation delay of 10 s, 1.69 s acquisition time, 128 K time domain data, 35878 Hz spectra width and typical 6000 repetitions were employed. Data was processed with 5 Hz line broadening.

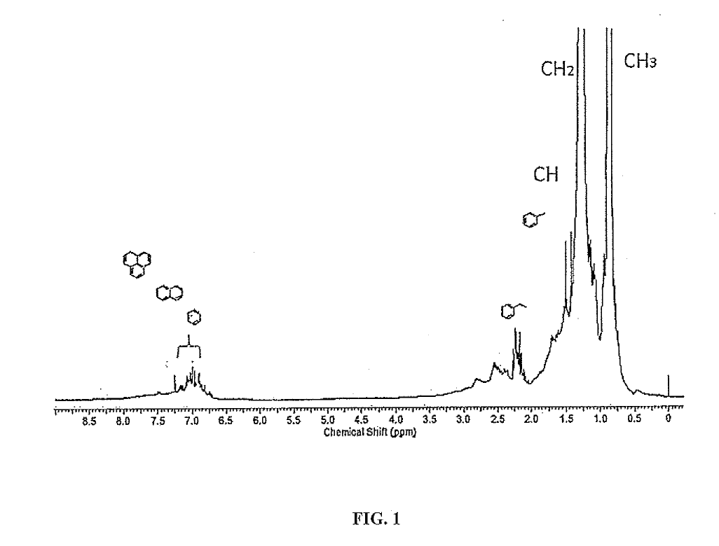

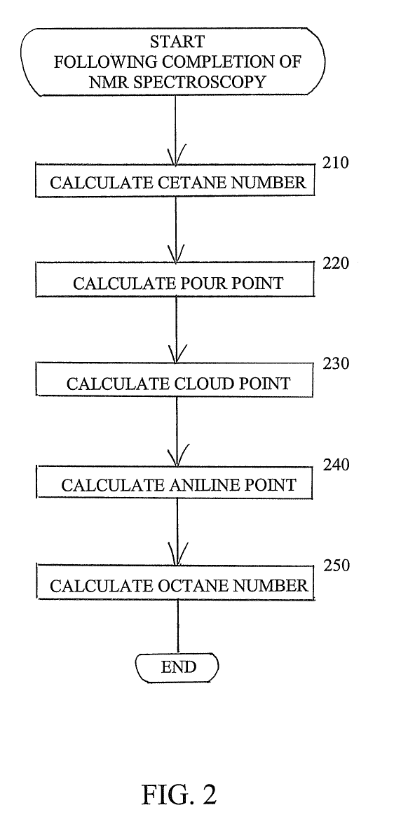

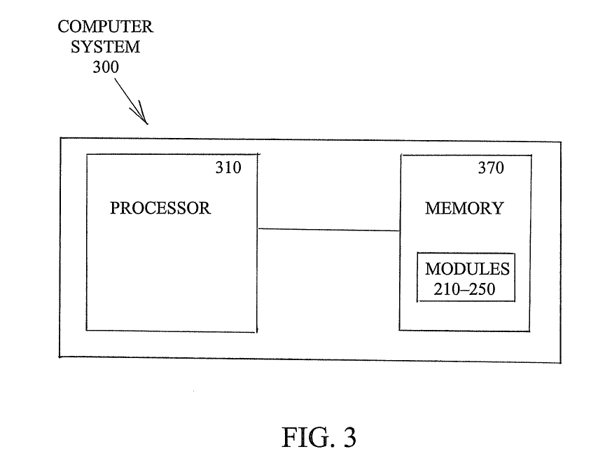

¹³C NMR spectra were obtained for all the oils and an example of the spectra is shown in FIG. 1. As seen in this FIGURE, the paraffinic, olefinic and aromatic carbons are identified on different regions of the spectra; the amounts of these carbons were determined by integrating the peaks identified. The carbon types were determined in the spectrum as follows:

Aromatic Region (165-100 ppm)

Aliphatic Region (75-5 ppm)

As for the paraffinic and naphthenic, the 75-5 ppm region of the spectrum is used to define integrals wherever a paraffin resonance is found. In this area total paraffinic carbons are determined. It is assumed that all narrow resonances are paraffinic, and that any obvious broader NMR peak groups that represent a superposition of narrow paraffinic resonances are 100% paraffinic. The naphthenic humps were removed from the spectrum first to determine the paraffinic carbons. The difference between the total paraffinic carbon and the paraffinic carbon determined the total naphthenic carbon.

As for ¹H NMR, the paraffinic and aromatic hydrogens were determined in the spectrum in the following regions:

| Hydrogen Type | Shift in Spectrum |
|---|---|
| Methyl (CH₃) protons of alkyl chains (γ) or further from the | 0.5-1.0 ppm |
| aromatic ring or methyl protons (CH₃) of saturated compounds | |
| (HS_{CH3}). | |
| Methylene (CH₂) and methane (CH) protons of alkyl chains (β) or | 1.00-2.00 ppm |
| further to ring and methyl (CH₃) protons (β) to the ring (HSβ + γ). | |
| Aromatic proton | 6.00-10.00 ppm |

### HS-Hydrogen Saturated

In a first embodiment when the only input is a ¹³C NMR spectra of crude oils, the indicative properties (i.e., the cetane number, pour point, cloud point, aniline point and octane number) of the gas oil fraction boiling in the range 180-370° C. can be predicted from the aromathic, naphthenic and paraffinic carbon content determined by ¹³C NMR spectra. That is,

Indicative Property=f(¹³C NMR Composition_{crude oil})

Equations (2) through (6) are detailed examples of this relationship.

Cetane Number (CET)=X1_{CET}*C_{N}+X2_{CET}*C_{P}+X3_{CET}*C_{A}+X4_{CET}*C_{N}²+X5_{CET}*C_{P}²+X6_{CET}*C_{A}² (2);

Pour Point (PP)=X1_{PP}*C_{N}+X2_{PP}*C_{P}+X3_{PP}*C_{A}+X4_{PP}*C_{N}²+X5_{PP}*C_{P}²+X6_{PP}*C_{A}² (3);

Cloud Point (CP)=X1_{CP}*C_{N}+X2_{CP}*C_{P}+X3_{CP}*C_{A}+X4_{CP}*C_{N}²+X5_{CP}*C_{P}²+X6_{CP}*C_{A}² (4);

Aniline Point (AP)=X1_{AP}*C_{N}+X2_{AP}*C_{P}+X3_{AP}*C_{A}+X4_{AP}*C_{N}²+X5_{AP}*C_{P}²+X6_{AP}*C_{A}² (5);

Octane Number (RON)=X1_{RON}*C_{N}+X2_{RON}*C_{P}+X3_{RON}*C_{A}+X4_{RON}*C_{N}²+X5_{RON}*C_{P}²+X6_{RON}*C_{A}² (6);

where:

C_{N}=¹H NMR-CH₃ protons of alkyl chains γ or further from aromatic ring or CH₃ of saturated compounds (HSCH3);

C_{P}=¹H NMR-CH₂ & CH protons of alkyl chains β or further to ring and CH₃ protons the ring (HSβ+γ);

C_{A}=¹H NMR-Aromatic H; and

X1_{CET}-X6_{CET}, X1_{PP}-X6_{PP}, X1_{CP}-X6_{CP}, X1_{AP}-X6_{AP}, and X1_{RON}-X6_{RON} are constants that were developed using linear regression techniques, and which are given in Table 3.

TABLE 3

| | Cetane | Pour | Cloud | Aniline | Octane |
|---|---|---|---|---|---|
| | Number | Point | Point | Point | Number |
| Property | (CET) | (PP) | (CP) | (AP) | (RON) |
| X1 | −843.8 | −1340.0 | −797.2 | −483.6 | 1196.0 |
| X2 | 744.0 | 420.7 | 32.2 | 368.5 | −940.8 |
| X3 | 381.6 | 2053.9 | 1792.0 | 723.7 | 373.5 |
| X4 | 1149.6 | 1729.9 | 1045.6 | 699.9 | −1561.5 |
| X5 | −808.7 | −532.4 | −84.6 | −378.6 | 1075.1 |
| X6 | −954.5 | −6502.8 | −5639.8 | −2207.0 | −964.7 |

In a second embodiment when density is considered in addition to a ¹³C NMR spectra of crude oils, the indicative properties (i.e., the cetane number, pour point, cloud point, aniline point and octane number) of the gas oil fraction boiling in the range 180-370° C. can be predicted from the whole crude oil density and aromathic, naphthenic and paraffinic carbon content determined by ¹³C NMR spectra. That is,

Indicative Property=f(density_{crude oil},¹³C NMR Composition_{crude oil}) (7);

Equations (8) through (12) are detailed examples of this relationship.

Cetane Number (CET)=X1_{CET}*DEN+X2_{CET}*C_{N}+X3_{CET}*C_{P}+X4_{CET}*C_{A}+X5_{CET}*C_{N}²+X6_{CET}*C_{P}²+X7_{CET}*C_{A}² (8);

Pour Point (PP)=X1_{PP}*DEN+X2_{PP}*C_{N}+X3_{PP}*C_{P}+X4_{PP}*C_{A}+X5_{PP}*C_{N}²+X6_{PP}*C_{P}²+X7_{PP}*C_{A}² (9);

Cloud Point (CP)=X1_{CP}*DEN+X2_{CP}*C_{N}+X3_{CP}*C_{P}+X4_{CP}*C_{A}+X5_{CP}*C_{N}²+X6_{CP}*C_{P}²+X7_{CP}*C_{A}² (10);

Aniline Point (AP)=X1_{AP}*DEN+X2_{AP}*C_{N}+X3_{AP}*C_{P}+X4_{AP}*C_{A}+X5_{AP}*C_{N}²+X6_{AP}*C_{P}²+X7_{AP}*C_{A}² (11);

Octane Number (RON)=X1_{RON}*DEN+X2_{RON}*C_{N}+X3_{RON}*C_{P}+X4_{RON}*C_{A}+X5_{RON}*C_{N}²+X6_{RON}*C_{P}²+X7_{RON}*C_{A}² (12);

where C_{N}, C_{P}, and C_{A} are as defined before;

DEN=density of the samples; and

constants X1_{CET}-X7_{CET}, X1_{PP}-X7_{PP}, X1_{CP}-X7_{CP}, X1_{AP}-X7_{AP}, and X1_{RON}-X7_{RON} are given in Table 4.

TABLE 4

| | Cetane | Pour | Cloud | Aniline | Octane |
|---|---|---|---|---|---|
| | Number | Point | Point | Point | Number |
| Property | (CET) | (PP) | (CP) | (AP) | (RON) |
| X1 | −112.8 | −213.5 | −125.9 | −91.0 | −277.5 |
| X2 | −672.8 | −1016.4 | −606.3 | −345.6 | 1562.4 |
| X3 | 995.0 | 895.7 | 312.4 | 571.0 | −321.2 |
| X4 | −282.1 | 798.0 | 1051.1 | 188.1 | −1130.1 |
| X5 | 1078.4 | 1595.2 | 966.1 | 642.5 | −1664.9 |
| X6 | −945.2 | −790.8 | −236.9 | −488.8 | 734.0 |
| X7 | 1509.4 | −1840.3 | −2889.4 | −218.6 | 4692.3 |

In a third embodiment when the only input is a ¹H NMR spectra of crude oils, the indicative properties (i.e., the octane number, pour point, cloud point, aniline point and octane number) of the gas oil fraction boiling in the range 180-370° C. can be predicted from the aromathic, naphthenic and paraffinic carbon content determined by ¹H NMR spectra. That is,

Indicative Property=f(¹H NMR Composition_{crude oil}) (13);

Equations (2) through (6) can be applied as detailed examples of this relationship, where C_{N}, C_{P}, and C_{A} are as defined before, and constants X1_{CET}-X6_{CET}, X1_{PP}-X6_{PP}, X1_{CP}-X6_{CP}, X1_{AP}-X6_{AP}, and X1_{RON}-X6_{RON} are given in Table 5.

TABLE 5

| | Cetane | Pour | Cloud | Aniline | Octane |
|---|---|---|---|---|---|
| | Number | Point | Point | Point | Number |
| Property | (CET) | (PP) | (CP) | (AP) | (RON) |
| X1 | −626.8 | −4361.5 | −2140.8 | −620.3 | 2504.3 |
| X2 | −2545.8 | −2815.3 | −3317.9 | −38.7 | −8517.3 |
| X3 | 37798.5 | 56783.6 | 50969.3 | 6716.1 | 84573.1 |
| X4 | 692.8 | 7448.9 | 3728.6 | 931.3 | −3537.2 |
| X5 | 2372.4 | 2888.7 | 3172.0 | 139.7 | 7837.1 |
| X6 | −415665.2 | −625842.1 | −561527.6 | −79178.8 | −921508.7 |

In a fourth embodiment when density is considered in addition to a ¹H NMR spectra of crude oils, the indicative properties (i.e., the cetane number, pour point, cloud point, aniline point and octane number) of the gas oil fraction boiling in the range 180-370° C. can be predicted from the whole crude oil density and aromathic, naphthenic and paraffinic carbon content determined by ¹H NMR spectra. That is,

Indicative Property=f(density_{crude oil},¹H NMR Composition_{crude oil}) (14);

Equations (8) through (12) can be applied as detailed examples of this relationship, where C_{N}, C_{P}, C_{A} and DEN are as defined before, and constants X1_{CET}-X7_{CET}, X1_{PP}-X7_{PP}, X1_{CP}-X7_{CP}, X1_{AP}-X7_{AP}, and X1_{RON}-X7_{RON} are given in Table 6.

TABLE 6

| | Cetane | Pour | Cloud | Aniline | Octane |
|---|---|---|---|---|---|
| | Number | Point | Point | Point | Number |
| Property | (CET) | (PP) | (CP) | (AP) | (RON) |
| X1 | −399.0 | −332.0 | −174.4 | −436.0 | −233.8 |
| X2 | −3093.2 | −6414.2 | −3218.8 | −3315.4 | −465.4 |
| X3 | 4465.7 | 3020.0 | −253.5 | 7622.9 | −5649.6 |
| X4 | −10114.5 | 16908.0 | 30028.9 | −45639.7 | 81342.3 |
| X5 | 4191.5 | 10360.7 | 5257.7 | 4754.4 | 1038.7 |
| X6 | −4177.3 | −2562.3 | 309.4 | −7017.3 | 5163.5 |
| X7 | 107503.5 | −190434.4 | −332876.3 | 492501.2 | −890961.9 |

The following example is provided. A sample of Arabian medium crude with a 15° C./4° C. density of 0.8828 Kg/1 was analyzed by ¹³C NMR spectroscopy. The crude oil fractional weight composition is 0.279 naphthenic, 0.529 paraffinic, and 0.192 aromatic carbon.

Applying equation (8) and the constants from Table 4,

$$Cetane Number ( CET ) = X {1}_{CET} * DEN + X {2}_{CET} * {C}_{N} + X {3}_{CET} * {C}_{P} + X {4}_{CET} * {C}_{A} + X {5}_{CET} * {C}_{N}^{2} + X {6}_{CET} * {C}_{P}^{2} + X {7}_{CET} * {C}_{A}^{2} = ( - 112.8 ) ( 0.8828 ) + ( - 672.8 ) ( 0.279 ) + ( 995.0 ) ( 0.529 ) + ( - 282.1 ) ( 0.192 ) + ( 1078.4 ) {( 0.279 )}^{2} + ( - 945.2 ) {( 0.529 )}^{2} + ( 1509.4 ) {( 0.192 )}^{2} = 60$$

Applying equation (9) and the constants from Table 4,

$$Pour Point ( PP ) = X {1}_{PP} * DEN + X {2}_{PP} * {C}_{N} + X {3}_{PP} * {C}_{P} + X {4}_{PP} * {C}_{A} + X {5}_{PP} * {C}_{N}^{2} + X {6}_{PP} * {C}_{P}^{2} + X {7}_{PP} * {C}_{A}^{2} = ( - 213.5 ) ( 0.8828 ) + ( - 1016.4 ) ( 0.279 ) + ( 895.7 ) ( 0.529 ) + ( 798.0 ) ( 0.192 ) + ( 1595.2 ) {( 0.279 )}^{2} + ( - 790.8 ) {( 0.529 )}^{2} + ( - 1840.3 ) {( 0.192 )}^{2} = - 10 ° C .$$

Applying equation (10) and the constants from Table 4,

$$Cloud Point ( CP ) = X {1}_{CP} * DEN + X {2}_{CP} * {C}_{N} + X {3}_{CP} * {C}_{P} + X {4}_{CP} * {C}_{A} + X {5}_{CP} * {C}_{N}^{2} + X {6}_{CP} * {C}_{P}^{2} + X {7}_{CP} * {C}_{A}^{2} = ( - 125.9 ) ( 0.8828 ) + ( - 606.3 ) ( 0.279 ) + ( 312.4 ) ( 0.529 ) + ( 1051.1 ) ( 0.192 ) + ( 966.1 ) {( 0.279 )}^{2} + ( - 236.9 ) {( 0.529 )}^{2} + ( - 2889.4 ) {( 0.192 )}^{2} = - 11 ° C .$$

Applying equation (11) and the constants from Table 4,

$$Aniline Point ( AP ) = X {1}_{AP} * DEN + X {2}_{AP} * {C}_{N} + X {3}_{AP} * {C}_{P} + X {4}_{AP} * {C}_{A} + X {5}_{AP} * {C}_{N}^{2} + X {6}_{AP} * {C}_{P}^{2} + X {7}_{AP} * {C}_{A}^{2} = ( - 91.0 ) ( 0.8828 ) + ( - 345.6 ) ( 0.279 ) + ( 571.0 ) ( 0.529 ) + ( 188.1 ) ( 0.192 ) + ( 642.5 ) {( 0.279 )}^{2} + ( - 488.8 ) {( 0.529 )}^{2} + ( - 218.6 ) {( 0.192 )}^{2} = 67 ° C .$$

Applying equation (12) and the constants from Table 4,

$$Octane Number ( RON ) = X {1}_{RON} * DEN + X {2}_{RON} * {C}_{N} + X {3}_{RON} * {C}_{P} + X {4}_{RON} * {C}_{A} + X {5}_{RON} * {C}_{N}^{2} + X {6}_{RON} * {C}_{P}^{2} + X {7}_{RON} * {C}_{A}^{2} = ( - 277.5 ) ( 0.8828 ) + ( 1562.4 ) ( 0.279 ) + ( - 321.2 ) ( 0.529 ) + ( - 1130.1 ) ( 0.192 ) + ( - 1664.9 ) {( 0.279 )}^{2} + ( 734.0 ) {( 0.529 )}^{2} + ( 4692.3 ) {( 0.192 )}^{2} = 53$$

The method is applicable for naturally occurring hydrocarbons derived from crude oils, bitumens, heavy oils, shale oils and from refinery process units including hydrotreating, hydroprocessing, fluid catalytic cracking, coking, and visbreaking or coal liquefaction.

The system and method of the present invention have been described above and with reference to the attached FIGURE; however, modifications will be apparent to those of ordinary skill in the art and the scope of protection for the invention is to be defined by the claims that follow.

1. A system for evaluating a crude oil sample and calculating indicative properties of a naphtha or gas oil fraction of the crude oil sample without first distilling the said naphtha or gas oil fraction, the system comprising:
an NMR spectroscope;
a non-volatile memory device that stores calculation modules and data;
a processor coupled to the non-volatile memory;
a first calculation module that calculates the cetane number for the gas oil fraction of the crude oil as a function of aromatic, naphthenic, and paraffinic carbon contents determined by NMR spectroscopy of the crude oil sample;
a second calculation module that calculates the pour point for the gas oil fraction of the crude oil as a function of aromatic, naphthenic, and paraffinic carbon contents determined by NMR spectroscopy of the crude oil sample;
a third calculation module that calculates the cloud point for the gas oil fraction of the crude oil as a function of aromatic, naphthenic, and paraffinic carbon contents determined by NMR spectroscopy of the crude oil sample;
a fourth calculation module that calculates the aniline point for the gas oil fraction of the crude oil as a function of aromatic, naphthenic, and paraffinic carbon contents determined by NMR spectroscopy of the crude oil sample; and
a fifth calculation module that calculates the octane number for the naphtha fraction of the crude oil as a function of aromatic, naphthenic, and paraffinic carbon contents determined by NMR spectroscopy of the crude oil sample.

2. The system of claim 1 in which the NMR spectroscopy employs ¹H active nuclei to determine the aromatic, naphthenic and paraffinic carbon contents.

3. The system of claim 1 in which the NMR spectroscopy employs ¹³C active nuclei to determine the aromatic, naphthenic and paraffinic carbon contents.

4. The system of claim 1 wherein the gas oil boils in the nominal range 180-370° C.

5. The system of claim 1 wherein the naphtha boils in the nominal range 36-180° C.

6. A system for evaluating a crude oil sample and calculating indicative properties of a naphtha or gas oil fraction of the crude oil sample without first distilling the said naphtha or gas oil fraction, the system comprising:
an NMR spectroscope;
a non-volatile memory device that stores calculation modules and data;
a processor coupled to the non-volatile memory;
a first calculation module that calculates the cetane number for the gas oil fraction of the crude oil as a function of the density of the crude oil sample and aromatic, naphthenic, and paraffinic carbon contents determined by NMR spectroscopy of the crude oil sample;
a second calculation module that calculates the pour point for the gas oil fraction of the crude oil as a function of the density of the crude oil sample and aromatic, naphthenic, and paraffinic carbon contents determined by NMR spectroscopy of the crude oil sample;
a third calculation module that calculates the cloud point for the gas oil fraction of the crude oil as a function of the density of the crude oil sample and aromatic, naphthenic, and paraffinic carbon contents determined by NMR spectroscopy of the crude oil sample;
a fourth calculation module that calculates the aniline point for the gas oil fraction of the crude oil as a function of the density of the crude oil sample and aromatic, naphthenic, and paraffinic carbon contents determined by NMR spectroscopy of the crude oil sample; and
a fifth calculation module that calculates the octane number for the naphtha fraction of the crude oil as a function of the density of the crude oil sample and aromatic, naphthenic, and paraffinic carbon contents determined by NMR spectroscopy of the crude oil sample.

7. The system of claim 6 in which the NMR spectroscopy employs ¹H active nuclei to determine the aromatic, naphthenic and paraffinic carbon contents.

8. The system of claim 6 in which the NMR spectroscopy employs ¹³C active nuclei to determine the aromatic, naphthenic and paraffinic carbon contents.

9. The system of claim 6 wherein the gas oil boils in the nominal range 180-370° C.

10. The system of claim 6 wherein the naphtha boils in the nominal range 36-180° C.

11. A method for evaluating a crude oil sample to determine indicative properties of a naphtha or gas oil fraction of the crude oil sample sample without first distilling the said naphtha or gas oil fraction, the method comprising:
subjecting said sample to NMR analysis;
calculating the cetane number for the gas oil fraction of the crude oil as a function of aromatic, naphthenic, and paraffinic carbon contents determined by the NMR analysis of the crude oil sample;
calculating the pour point for the gas oil fraction of the crude oil as a function of aromatic, naphthenic, and paraffinic carbon contents determined by the NMR analysis of the crude oil sample;
calculating the cloud point for the gas oil fraction of the crude oil as a function of aromatic, naphthenic, and paraffinic carbon contents determined by the NMR analysis of the crude oil sample;
calculating the aniline point for the gas oil fraction of the crude oil as a function of aromatic, naphthenic, and paraffinic carbon contents determined by the NMR analysis of the crude oil sample; and
calculating the octane number for the naphtha fraction of the crude oil as a function of aromatic, naphthenic, and paraffinic carbon contents determined by the NMR analysis of the crude oil sample.

12. The method of claim 11 in which the NMR spectroscopy employs ¹H active nuclei to determine the aromatic, naphthenic and paraffinic carbon contents.

13. The method of claim 11 in which the NMR spectroscopy employs ¹³C active nuclei to determine the aromatic, naphthenic and paraffinic carbon contents.

14. The method of claim 11 wherein the gas oil boils in the nominal range 180-370° C.

15. The method of claim 11 wherein the naphtha boils in the nominal range 36-180° C.

16. A method for evaluating a crude oil sample to determine indicative properties of a naphtha or gas oil fraction of the crude oil sample sample without first distilling the said naphtha or gas oil fraction, the method comprising:
determining density of the crude oil sample;
subjecting said crude oil sample to NMR analysis;
calculating the cetane number for the gas oil fraction of the crude oil as a function of the density of the crude oil sample and aromatic, naphthenic, and paraffinic carbon contents determined by the NMR analysis of the crude oil sample;
calculating the pour point for the gas oil fraction of the crude oil as a function of the density of the crude oil sample and aromatic, naphthenic, and paraffinic carbon contents determined by the NMR analysis of the crude oil sample;
calculating the cloud point for the gas oil fraction of the crude oil as a function of the density of the crude oil sample and aromatic, naphthenic, and paraffinic carbon contents determined by the NMR analysis of the crude oil sample;
calculating the aniline point for the gas oil fraction of the crude oil as a function of the density of the crude oil sample and aromatic, naphthenic, and paraffinic carbon contents determined by the NMR analysis of the crude oil sample; and
calculating the octane number for the naphtha fraction of the crude oil as a function of the density of the crude oil sample and aromatic, naphthenic, and paraffinic carbon contents determined by the NMR analysis of the crude oil sample.

17. The method of claim 16 in which the NMR spectroscopy employs ¹H active nuclei to determine the aromatic, naphthenic and paraffinic carbon contents.

18. The method of claim 16 in which the NMR spectroscopy employs ¹³C active nuclei to determine the aromatic, naphthenic and paraffinic carbon contents.

19. The method of claim 16 wherein the gas oil boils in the nominal range 180-370° C.

20. The method of claim 16 wherein the naphtha boils in the nominal range 36-180° C.

21. A system for evaluating a crude oil sample and calculating an indicative property of a naphtha or gas oil fraction of the crude oil without first distilling the said naphtha or gas oil fraction, the system comprising:
an NMR spectroscope;
a non-volatile memory device that stores calculation modules and data;
a processor coupled to the non-volatile memory;
a calculation module that calculates the indicative property for the gas oil or naphtha fraction of the crude oil as a function of aromatic, naphthenic, and paraffinic carbon contents determined by NMR spectroscopy of the crude oil sample.

22. The system of claim 21, wherein the indicative property is the cetane number.

23. The system of claim 21, wherein the indicative property is the pour point.

24. The system of claim 21, wherein the indicative property is the cloud point.

25. The system of claim 21, wherein the indicative property is the aniline point.

26. The system of claim 21, wherein the indicative property is the octane number.

27. A system for evaluating a crude oil sample and calculating an indicative property of a naphtha or gas oil fraction of crude oil without first distilling the said naphtha or gas oil fraction, the system comprising:
an NMR spectroscope;
a non-volatile memory device that stores calculation modules and data;
a processor coupled to the memory;
a calculation module that calculates the indicative property for the gas oil or naphtha fraction of the crude oil as a function of density of the crude oil sample and aromatic, naphthenic, and paraffinic carbon contents determined by NMR spectroscopy of the crude oil sample.

28. The system of claim 27, wherein the indicative property is the cetane number.

29. The system of claim 27, wherein the indicative property is the pour point.

30. The system of claim 27, wherein the indicative property is the cloud point.

31. The system of claim 27, wherein the indicative property is the aniline point.

32. The system of claim 27, wherein the indicative property is the octane number.

33. A method for evaluating a crude oil sample to determine an indicative property of a naphtha or gas oil fraction of the crude oil sample without first distilling the said naphtha or gas oil fraction, the method comprising:
subjecting said sample to NMR analysis;
calculating the indicative property for the gas oil or naphtha fraction of the crude oil as a function of aromatic, naphthenic, and paraffinic carbon contents determined by the NMR analysis of the crude oil sample.

34. The method of claim 33, wherein the indicative property is the cetane number.

35. The method of claim 33, wherein the indicative property is the pour point.

36. The method of claim 33, wherein the indicative property is the cloud point.

37. The method of claim 33, wherein the indicative property is the aniline point.

38. The method of claim 33, wherein the indicative property is the octane number.

39. A method for evaluating a crude oil sample to determine an indicative property of a naphtha or gas oil fraction of the crude oil sample without first distilling the said naphtha or gas oil fraction, the method comprising:
determining density of the crude oil sample;
subjecting said sample to NMR analysis;
calculating the indicative property for the gas oil or naphtha fraction of the crude oil as a function of the density of the crude oil sample and aromatic, naphthenic, and paraffinic carbon contents determined by the NMR analysis of the crude oil sample.

40. The method of claim 39, wherein the indicative property is the cetane number.

41. The method of claim 39, wherein the indicative property is the pour point.

42. The method of claim 39, wherein the indicative property is the cloud point.

43. The method of claim 39, wherein the indicative property is the aniline point.

44. The method of claim 39, wherein the indicative property is the octane number.
